(19) United States
(12) Patent Application Publication
(10) Pub. No.: US 2024/0252131 A1
(43) Pub. Date: Aug. 1, 2024

(54) DIGITAL DENTAL XRAY SENSOR, METHOD AND SYSTEM

(71) Applicant: DENTAL SMARTMIRROR, INC., Chicago, IL (US)

(72) Inventors: Gidon ODED ELAZAR, Cohav Yair (IL); Dan Zidkiahu HARKABI, Moshav Lachish (IL)

(73) Assignee: DENTAL SMARTMIRROR, INC., Chicago, IL (US)

(21) Appl. No.: 18/425,700

(22) Filed: Jan. 29, 2024

Related U.S. Application Data
(60) Provisional application No. 63/441,670, filed on Jan. 27, 2023.

(51) Int. Cl. A61B 6/51; A61B 6/00; A61B 6/42; A61B 6/46

(52) U.S. Cl. CPC A61B 6/512; A61B 6/425; A61B 6/4452; A61B 6/488; A61B 6/547; A61B 6/462

(57) ABSTRACT

A digital dental x-ray sensor and methods to align the digital dental x-ray sensor are disclosed herein. The digital dental x-ray sensor includes an autoclavable portion which includes an appendage and an x-ray sensor head including an x-ray capturing element, a camera, and an inertial measurement unit. Additionally, the digital dental x-ray sensor includes a non-autoclavable portion which includes a connector, a processor, and a power supply.

### BACKGROUND

### Field

The present disclosure relates to digital x-ray sensors in dental health care.

### Background

Dental x-ray images, or radiographs, are images created by capturing or sensing x-ray radiation transmitted through a section of the mouth or oral cavity. Since x-ray radiation may pass through tissue, it can be effectively used to capture the shape of a patient's teeth, including parts that are embedded in the patient's bones, such as roots of teeth. This makes x-ray images particularly useful for dental healthcare practitioners to diagnose the condition of a patient's mouth, and thus are frequently included in the determination of a required treatment.

To capture x-ray images, dental practitioners will place an x-ray capturing device, for example an x-ray film, a phosphor plate, or an x-ray digital sensor, inside the patient's mouth close to its lingual surface (that is, for example, between the teeth and the tongue). Then they would bring the emitting end of an x-ray emitter, sometimes referred to as x-ray tube head or cannon, towards the patient's face, closer to the exterior of the patient's mouth or cheek and aiming towards the center of the x-ray capturing device, usually in a position as perpendicular to the x-ray capturing device as possible.

X-ray films are made of a flexible material, thus may bend, and have on one side a substance that chemically changes when exposed to x-ray radiation. The image is therefore imprinted on the film thus these films enable a single image capture. That is a major reason that for many practitioners this type of capturing device is not the device of choice at present.

X-ray phosphor plates are also thin and flexible, similarly to the film, but unlike film, they are coated with a material that absorbs energy when exposed to x-ray radiation, in a way storing energy in the locations exposed to x-ray. The energy can be later released, for example as visible light, when illuminated. Thus after capturing an x-ray image, and extracting the plate from the patient's mouth, the plate has to be processed in a plate reader. The reader scans the plate with illumination, for example using a laser, eventually extracting the image, a process that spans over several tens of seconds up to about a minute. Illumination also releases the stored energy, which in any case decays with time, preparing the plate for the next cycle, thus the plates may be used multiple times for capturing many x-ray images. For that reason they are considered an improved replacement to film.

Several steps are involved in capturing an x-ray image with a phosphor plate. Prior to placing a plate in a patient's mouth, the plate is inserted into a single-use envelope that serves as a barrier between the multi-use plate and the tissue. The enveloped plate then needs to be positioned in the area of interest inside the mouth. Then, an x-ray tube has to be positioned in proximity to the patient's face, pointing perpendicularly towards the center of the plate. The plate may be secured in place, in order to keep it in a fixed position inside the mouth. In many cases, the patient bites on a structure that supports the plate, usually made of plastic. For example, the plate may be inserted into a bitewing holder, a structure that holds the plate and has a wing-like extension, named bitewing, for the patient to bite on. When inside the mouth, the plate is usually not clearly visible to the practitioner, making it challenging to adjust the location of the x-ray tube head. To assist this process, plates are sometimes placed inside a device, called a positioner, prior to being inserted into a patient's mouth. The positioner is usually made of plastic and it extends outside of the mouth and then back towards the check so that it ends near the face of the patient, and parallel to the plate inside. A plastic circle at the positioner end marks the area where the tube head should aim. Unfortunately, the positioners are cumbersome to use and not always provide the intended result. For example, the geometry required from the positioner varies between anterior or posterior teeth, requiring the practitioner to either use different positioners or adjust the positioner and the plate to a correct geometry prior to use. While usage of a positioner provides the practitioner with indication for aiming the tube, the indication is not precise and it is easily feasible to position the tube within the area of the plastic circle, but still miss the center of the sensor or the perpendicular alignment. These make the usage of a positioner to require discipline of the practitioner. To compensate for these issues, the area of the patient's face exposed to x-ray radiation tends to be larger than the area of the plate, thus the plate will be exposed to x-ray radiation even when there is some limited error in positioning. Despite this process being lengthy, many practitioners follow it in order to improve the quality of the captured images. Digital x-ray sensors have an x-ray capturing element that includes a digital image sensor, which may be similar to an image sensor of a digital camera, such as a CMOS sensor, which captures light and generates a digital image. For converting x-ray radiation into light, the x-ray capturing element has a component named scintillator, a layer parallel to the digital image sensor that emits light when exposed to x-ray radiation, where the area of the scintillator that has been exposed to x-ray is the area emitting the light. The emitted light from the scintillator area is then funneled to the respective cells of the image sensor where it is captured and eventually a digital image is created. The image is therefore available instantly, similarly to a digital image captured in a digital camera. Digital x-ray sensors require power to function, and a communications method to transfer the captured image. Many digital x-ray sensors have a digital sensor head with the x-ray capturing element attached to a computer through a cable, usually a USB cable. The cable connects the head to a computer where the image will eventually be displayed.

There is a health risk involved with human exposure to x-ray radiation, and it may depend on the dose of exposure, patient's age, sex, the area exposed and more. The risk is not only to the patient, but also to the health care provider, which may be exposed much more frequently to such radiation, even if indirectly. To reduce the risk, a health care provider may, for example, leave the room before engaging the x-ray emitter, and take cover behind a protecting wall while x-ray transmission is in progress. In contrast, handheld x-ray units are increasingly available, and these require a practitioner to physically hold them in the required position while an x-ray image is being captured.

Because of the associated risk, when the capturing of x-ray images is required, practitioners will attempt to expose the patient to the least amount of radiation possible. One of the variables affecting the amount of radiation that the patient is exposed to are the settings of the x-ray emitter when capturing a single x-ray image. Since objects in a patient's mouth have varying structure components, bone, tooth, softer tissue etc., they may also have varying requirements for exposure for producing a good enough x-ray image. For example, to capture an image of an object that has a thicker bone may require more exposure to x-ray than required by an object with mostly soft tissues. To enable the practitioner to control the exposure, many x-ray emitters allow setting the exposure time. When higher exposure to x-ray is desired, the setting is turned to a longer exposure time, and for less exposure to x-ray, the setting is turned to lower exposure time.

There are other possible settings that can affect the amount of x-ray exposure by an x-ray emitter, such as the internal voltage that produces potential difference or such as the current feed, which may affect the amount of radiation per time measurement, but those are, in many cases, not available for the operator to modify, thus providing a simpler operation which is therefore less error prone.

There is one additional important variable that affects the total x-ray dose that a patient may be exposed to, which is the number of x-ray images captured. It is therefore important that a resulting image will indeed contain the information that the practitioner aimed to capture and at the desired quality, otherwise, repeating the process may be necessary, exposing the patient to more radiation. An image may fail to include the information that the practitioner aimed to capture if, for example, while setting up the scene the capturing device was positioned incorrectly inside the patient's mouth, or, for example, if the x-ray tube head was not positioned correctly with respect to the capturing device, or, as another example, if the patient moves after the setup of the scene.

The required exposure time for capturing an image is affected by the tissue, the age of the patient, and also by the capturing device where the more sensitive to x-ray radiation the device is, the less exposure it requires. Generally, advancement in digital x-ray sensors makes them more sensitive than phosphor plates, sometimes substantially more sensitive, thus may require considerably less exposure for each x-ray image capture. Normally, the operator will set the exposure time to a value between 0.16 to 0.40 seconds for phosphor plates and 0.06 to 0.16 seconds for a digital sensor.

Unfortunately, at present, digital x-ray sensors are more expensive than plates, and also more difficult to position correctly inside a patient's mouth and subsequently to successfully complete an x-ray image capture. This is due to several reasons. First, there is electronics embedded inside the digital x-ray sensors that makes the sensor bulkier than a phosphor plate. Second, a plate has a certain degree of flexibility, and may adapt its geometry to the mouth of the patient; a digital sensor, in contrast, is rigid. Third, the outer part of a digital sensor is an encasing, possibly made of plastic, protecting its internal components, thus the external dimensions of the x-ray sensor are larger than the actual x-ray sensitive area, ending, in practice, having a smaller effective area than in a comparable film. This means that a correct setup of the scene is more nuanced, and it is easier to incorrectly position the x-ray tube in respect to the sensor and to effectively capture x-ray images.

What is needed is a device, method and system that will allow dental practitioners to more easily and faster use a digital x-ray sensor for capturing x-ray images, and improve the process of aligning an x-ray tube with an x-ray sensor.

### SUMMARY

Described herein is a method for aligning a dental instrument. In some aspects, a trail set is captured from a first image sensor affixed to an x-ray sensor head, wherein the trail set comprises a plurality of images captured by the first image sensor, the plurality of images continuously captured as the x-ray sensor head is shifted from a trail origin position representing a starting point of the trail set to a pending position representing a desired position for an x-ray image of the patient's mouth to be captured. A respective position is then calculated for each image in the plurality of images based on the trail origin position.

In some aspects, a first image is captured from a first image sensor affixed to an x-ray sensor head, the first image captured at an origin position. A pending position of the x-ray sensor head is determined with reference to the origin position. It is determined whether the first image shares a common axis of reference with an x-ray tube based on a correlation between the first image and a second image captured by a second image sensor affixed to the x-ray tube. A position of the x-ray tube is calculated with reference to the pending position of the x-ray sensor head and the common axis of reference. Based on the position of the x-ray tube, it is determined whether the x-ray tube is perpendicularly aligned towards the x-ray sensor head when at the pending position.

In some aspects, an x-ray sensor device is handheld by a healthcare practitioner and placed inside a patient's mouth. The x-ray sensor device detects x-ray signals from an x-ray tube head. The x-ray tube and x-ray sensor each have or are coupled with cameras and inertial measurement units (IMU) that are used to determine their location and orientation with respect to each other. The application uses the determined location and orientation to direct the healthcare practitioner to position the tube head so it is aiming directly perpendicular at the center of the x-ray sensor. The application can continuously check the alignment and notify the practitioner if they go out of alignment. The IMUs can be calibrated using a base station and errors can be corrected using a Kalman filter.

In some aspects, an x-ray sensor device is handheld by a healthcare practitioner and placed inside a patient's mouth. The x-ray sensor device detects x-ray signals from an x-ray tube head. The x-ray sensor and x-ray tube head have markers. A camera captures both markers concurrently. Images from the camera are analyzed to detect the markers and determine alignment of the tube head with respect to the sensor. The application uses the determined alignment to direct the healthcare practitioner to position the x-ray tube head to aim directly perpendicular at the center of the x-ray sensor. The application can continuously check the alignment and notify the practitioner if they go out of alignment.

In some aspects, an x-ray sensor device is handheld by a healthcare practitioner and placed on a device inside a patient's mouth. The x-ray sensor device detects x-ray signals from an x-ray tube head. The x-ray sensor device includes one or more lights that illuminate through a patient's cheek. The healthcare practitioner uses the light to position the x-ray tube head to aim directly perpendicular at the center of the x-ray sensor.

In some aspects, an x-ray sensor is handheld by a healthcare practitioner and placed inside a patient's mouth. The x-ray sensor device detects x-ray signals from an x-ray tube head. The application notifies the healthcare practitioner if the tube head and sensor are aligned or not. The healthcare practitioner uses the indication to send a signal to capture the image, and the x-ray image is sent to the healthcare practitioner's mobile device display.

In some aspects, an x-ray sensor is handheld by a healthcare practitioner and placed inside a patient's mouth. The x-ray sensor device detects x-ray signals from an x-ray tube head. The x-ray sensor has or is coupled with an inertial measurement unit (IMU), lights and a camera. The lights and camera assist the practitioner in positioning the sensor correctly inside the mouth. Analysis of the camera and IMU data is used to alert the practitioner if the sensor shifted positions or the patient has moved thus risking that the capture of an x-ray image will fail to provide the desired results.

In some aspects, an x-ray sensor is handheld by a healthcare practitioner and placed inside a patient's mouth. The x-ray sensor device detects x-ray signals from an x-ray tube head. The x-ray sensor has or is coupled with an inertial measurement unit (IMU) and a camera. The data from the IMU and images from the camera are used to facilitate the stitching together of multiple X-ray images. Prior to the x-ray images being captured, data received from the IMU and the camera is used to determine whether the x-ray images will have sufficient overlap.

In some aspects, an x-ray sensor is handheld by a healthcare practitioner and placed inside a patient's mouth. The x-ray sensor device detects x-ray signals from a handheld x-ray tube head. The handheld x-ray tube may have a display. The application notifies the healthcare practitioner that the tube head and sensor are aligned. The healthcare practitioner sends a signal to capture the image, and the x-ray image may be sent to the handheld x-ray tube and shown on the display.

System and device aspects are also disclosed.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIGS. 1A-1D and 2A-2C illustrate a digital x-ray sensor device including an integrated camera, according to some aspects.

FIG. 3 is a flowchart illustrating a method for determining a position of a digital x-ray sensor inside a patient's mouth, according to some aspects.

FIG. 4A-4B illustrate systems and methods that utilize the digital x-ray sensor, according to some aspects.

FIG. 5 is a flowchart illustrating a method for using the x-ray sensor device to capture a patient's panoramic x-ray, according to some aspects.

FIGS. 6A-6C illustrate systems and methods for assisting the alignment of an x-ray sensor head and an x-ray tube head for capturing an x-ray image of a patient, according to some aspects.

FIGS. 7A and 7B illustrate alternative aspects of an x-ray sensor device.

FIGS. 8A-8E illustrate an x-ray sensor head coupled with lights that traverse the patient's tissue indicating the location of the sensor, according to some aspects.

FIG. 9 illustrates a block diagram of an x-ray sensor device, according to some aspects.

In the drawings, like reference numbers generally indicate identical or similar elements. Additionally, generally, the left-most digit(s) of a reference number identifies the drawing in which the reference number first appears.

Aspects of the present disclosure will be described with reference to the accompanying drawings.

### DETAILED DESCRIPTION

Digital x-Ray Sensor Including a Digital Camera

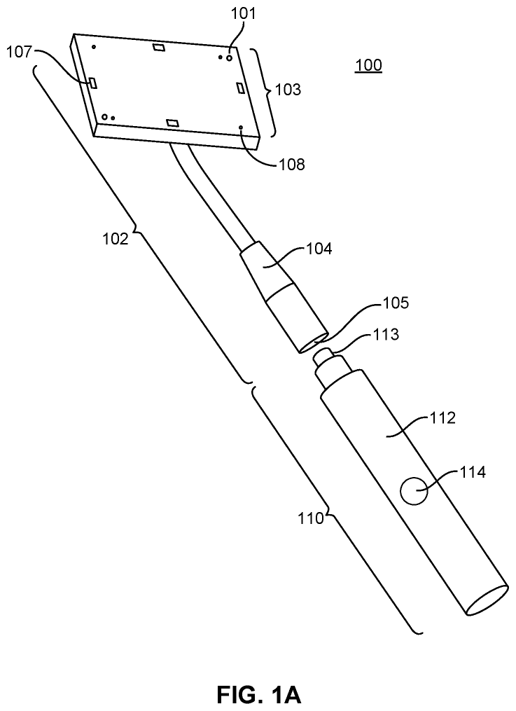

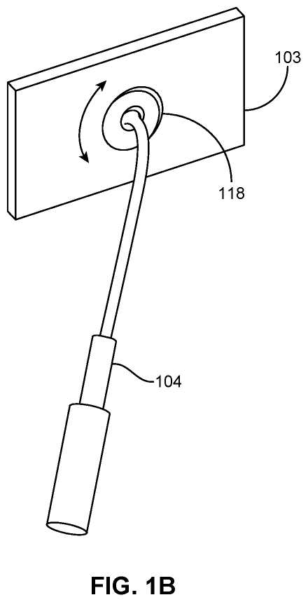

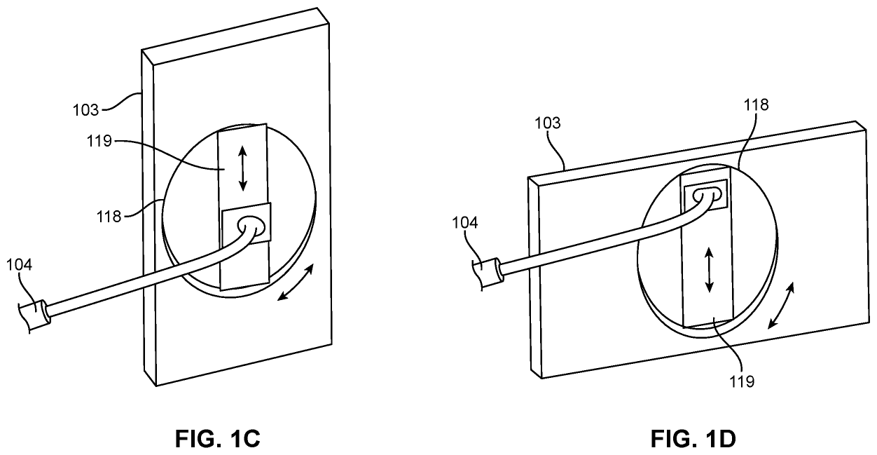

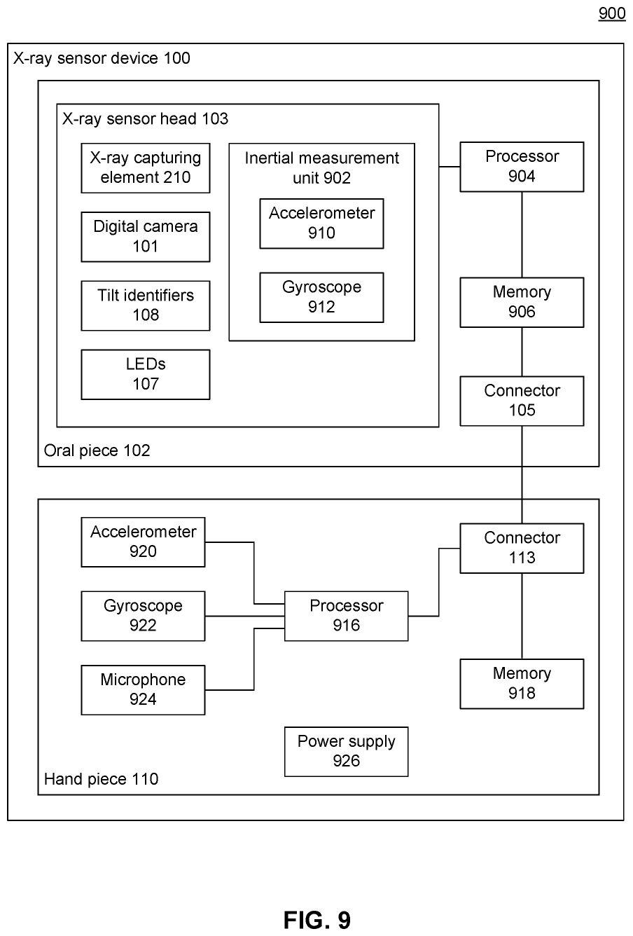

FIG. 1A illustrates components of a digital x-ray sensor including an integrated camera, according to an aspect. In particular, FIG. 1A illustrates physical components of x-ray sensor device 100. Additionally, FIG. 9 illustrates a block diagram of the digital x-ray sensor with respect to components illustrated in FIG. 1A.

X-ray sensor device 100 includes two separate, detachable physical components: an oral piece 102 and a hand piece 110. The hand and oral pieces 110 and 102 may be further detachable into sub-pieces. Oral piece 102 may be the portion of x-ray sensor device 100 that can enter a patient's mouth.

Oral piece 102 may include portions of the x-ray sensor device 100 that can be sterilized through being autoclaved. To be autoclaved, oral piece 102 can withstand being placed in an autoclave machine, which is a pressure chamber used to carry out industrial processes requiring elevated temperature and pressure different from ambient air pressure. Many autoclaves are used to sterilize equipment and supplies by subjecting them to high-pressure saturated steam at 121-132° C. (249-270° F.) for around 15-20 minutes depending on the size of the load and the contents, although autoclaving oral piece 102 using a lower pressure or temperature autoclaves, which at present are less commonly used, may increase the lifespan of oral piece 102. Additionally, oral piece 102 may be replaceable, such that one of oral piece 102 may be ongoing sterilization while another one is being used for x-ray image capture.

In contrast to oral piece 102, hand piece 110 is not adapted to be in contact with a patient's mouth. Because hand piece 110 is not in contact with a patient's mouth, it may not need to be sterilized in an autoclave like oral piece 102. Hand piece 110 may include portions of the smart mirror device 100 that cannot withstand being autoclaved. For example, hand piece 110 may include sensitive electronics and a power supply that could be damaged by the heat, pressure, and moisture in an autoclave machine. In some aspects, hand piece 110 may include a microphone (not shown) for capturing audio for example for the identification of voice commands. Also, components may be placed on hand piece 110 as opposed to oral piece 102 to avoid the need to replace them as they become worn out through repeated use and sterilization.

Returning to oral piece 102, oral piece 102 includes an x-ray sensor head 103, an appendage 104, and a connector 105. X-ray sensor head 103 has at least some of the electronics of oral piece 102. X-ray sensor head 103 includes an x-ray capturing element (internal, thus concealed and not shown in the diagram) and one or more camera 101 that have the ability to capture images or video. X-ray sensor head 103 may additionally have LEDs 107 that may provide illumination to practitioners while inserting oral piece 102 into a patient's mouth to improve their visibility, or to brighten the intra-oral environment for camera 101 when capturing intraoral images and video, thus potentially improving the quality of the resulting image. X-ray sensor head 103 may additionally have an inertial measurement unit 902 (as illustrated by FIG. 9), which contains accelerometers 910 and/or gyroscopes 912 to enable the calculation of the position or orientation of x-ray sensor device 100 and particularly of x-ray sensor head 103. X-ray sensor head 103 may additionally include a processor 904 and a memory 906 (as illustrated in FIG. 9) to store x-ray images.

Memory 906 may include random access memory (RAM) and may also include nonvolatile memory, such as read only memory (ROM) and/or flash memory. Memory 906 may be embodied as an independent memory component, and may also be embedded in another component, such as processor 904 and/or x-ray sensor head 103, or may be embodied as a combination of independent as well as embedded, and/or a plurality of memory components. Memory 906 is adapted to include software modules (a module is a set of instructions).

Processor 904 is adapted to run instructions stored in memory 906. Processor 904 may be a micro-controller unit (MCU), a digital signal processor (DSP) and/or an Image/Video Processing unit or like components that run instructions. An example of an MCU is MSP432P401x available from Texas Instruments Inc. of Dallas, Texas. An example of a DSP is C5000 available from Texas Instruments Inc. of Dallas, Texas. An example of an image/video processor is OMAP3525 also available from Texas Instruments Inc. of Dallas, Texas. Another example of the processor is the FPGA, such as the one available from Xilinx. One or more processor 904 may be present. Processor 904 may be an independent component, it may also be embedded in another component, such as in x-ray sensor head 103. Each subcomponent may also have internal memory devices.

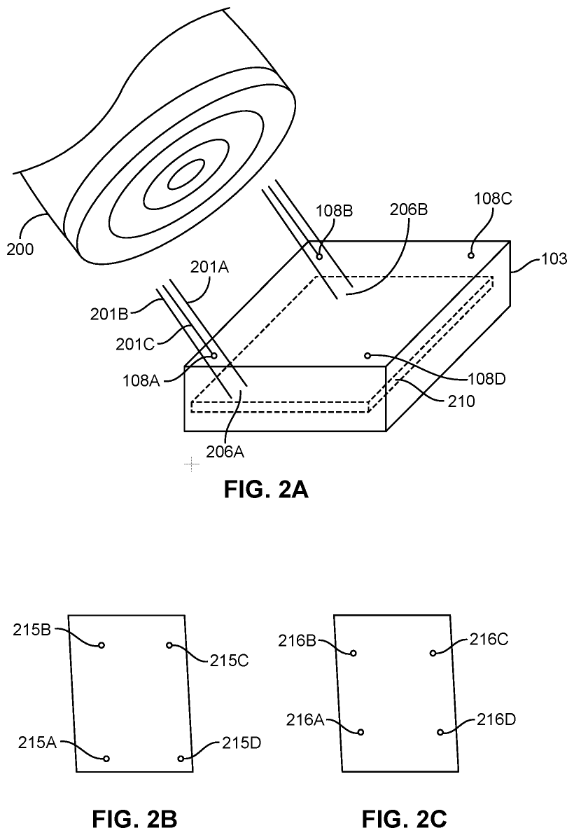

Additionally, x-ray sensor head 103 may have one or more tilt identifiers 108A-D. Tilt identifiers 108A-D are made of a material that obstructs x-ray radiation, for example lead. FIG. 2A is a diagram illustrating an aspect of x-ray sensor head 103 while being exposed to x-ray radiation emitted from a tilted x-ray tube 200 (in the diagram, depicted the emitting end of x-ray tube 200). Shown in the diagram, x-ray sensor head 103, including an x-ray capturing element 210, includes tilt identifiers 108A-D, which are respectively equidistant from the corners of x-ray capturing element 210. The illustration shows x-ray capturing element 210 in dotted lines as it is internal to x-ray sensor head 103, thus not normally viewed from outside. X-ray tube 200, shown in part, is emitting x-ray radiation towards x-ray sensor head 103, while being tilted in respect to x-ray sensor head 103 or more specifically to x-ray capturing element 210, thus the emitted rays arrive at an angle, and not perpendicular, to x-ray capturing element 210, 201A, 201B and 201C represent rays emitted from x-ray tube 200, which are substantially parallel to each other. Rays 201A and 201B are not obstructed and therefore reach x-ray capturing element 210, while ray 201C is obstructed by tilt identifier 108A and therefore do not reach x-ray capturing element 210. This will result in a shadow 206A of tilt identifier 108A on x-ray capturing element 210, an area that remains unexposed to x-ray radiation, therefore an x-ray image captured at this configuration will show a corresponding unexposed area, which may appear in an x-ray image as a bright white area. This similarly applies to tilt identifier 108B and shadow 206B. Since there is some distance between tilt identifiers 108A-D and x-ray capturing element 210, a tilt angle of x-ray tube 200 determines the position of shadows 206A-B on x-ray capturing element 210.

FIG. 2B depicts an aspect of an x-ray image captured at a configuration similar to shown in FIG. 2A, where some areas of x-ray capturing element 210 were obstructed by tilt identifiers 108A-D. In the diagram, depicted unexposed areas 215A-D corresponding to tilt identifiers 108A-D respectively. The locations of unexposed areas 215A-D are not respectively equidistant from the edges and corners of the x-ray image, which may indicate that the x-ray image was captured while x-ray tube 200 was tilted, thus rays 201A-C arrived at an angle to x-ray capturing element 210. In such x-ray images, the objects may appear as stretched or skewed, thus the image may seem distorted. Using unexposed areas 215A-D the image could be processed and transformed to produce a corrected image. FIG. 2C is an aspect of such transformation, where unexposed areas 215A-D were transformed to transformed areas 216A-D, where the ratio of distances between each of transformed areas 216A-D is kept similar to the ratio of distances between tilt identifiers 108A-D respectively.

Back to oral piece 102 in FIG. 1A, appendage 104 is affixed to x-ray sensor head 103, protecting hand piece 110 from contacting the intraoral environment while allowing for comfortable intraoral use of x-ray sensor head 103. Appendage 104 has an elongated shape with x-ray sensor head 103 affixed to one end of the shape, extending x-ray sensor head 103 into the patient's mouth. In some aspects, at least part of appendage 104 is thin enough to allow a patient to bite on, so that the biting holds appendage 104 and consequently x-ray sensor head 103 in a fixed position. In some aspects, at least parts of appendage 104 have a shape of a tube or cylinder, but other shapes, including those with non-rounded cross-sections, are possible. In aspects, at least part of appendage 104 is hollow, allowing space for electronic components.

FIG. 1B depicts an aspect of oral piece 102 from a different perspective. In some aspects, appendage 104 is affixed to a rotating element 118 attached to x-ray sensor head 103, at the back side of x-ray sensor head 103 (opposite to the side showing camera 101). Rotating element 118 allows x-ray sensor head 103 to rotate in respect to appendage 104, thus enabling a practitioner to change the angle between x-ray sensor head 103 to rotate in respect to appendage 104 to ease reaching and positioning x-ray sensor head 103 in a desired location in a patient's mouth for capturing an x-ray image. The angle of rotation rotating element 118 may be measured using electronic circuitry, for example, by measuring electric current or voltage at a sliding contact over a variable resistor, or, for example, by having a locking mechanism that locks the rotation at angles of interest, each closing a separate circuit indicating the locked angle.

Additional to the elements shown in FIG. 1B, FIGS. 1C-1D illustrate a slider element 119 attached to rotating element 118. Slider element 119 allows appendage 104 to be linearly displaced between one end of the slider and the other end. FIGS. 1C and 1D depict settings of a rotation of rotating element 118 and amount of displacement of slider element 119. The displacement of slider element 119 may be measured using electronic circuitry.

Back to FIG. 1A, while one end of appendage 104 is affixed to x-ray sensor head 103, the opposite end is affixed to a connector 105. Connector 105 is a physical and electrical connector to connect oral piece 102 with hand piece 110. In some aspects, connector 105 is internal to appendage 104. For example, hand piece 110 can slide or partially slide into a hollow part of appendage 104 to attach hand piece 110 with connector 105. An impermeable lid (not shown) may be used to seal off connector 105 when oral piece 102 is autoclaved. The lid may have a pressure valve, to enable the release of pressure if such may rise inside oral piece 102 when temperature rises. Appendage 104 may interlock with hand piece 110 to avoid separation of oral piece 102 and hand piece 110 during use of x-ray sensor device 100. To interlock with hand piece 110, the two pieces may, for example, screw into one another, or pressure from hand piece 110 sliding into appendage 104 may hold the two pieces together during use.

In some aspects where the oral piece and hand piece are connected, they form a shape that allows a practitioner to grasp x-ray sensor device 100 while an x-ray image is being captured, without exposure or with acceptable exposure to the x-ray emissions. In some aspects, x-ray sensor device 100 forms an elongated shape, long enough to create enough distance between the section being grasped and the x-ray emission source. In some aspects, x-ray sensor device 100 forms a shape that folds escaping the path of the x-ray radiation. In some aspects, a protective shield is attached (not shown), so that a barrier protecting the areas being grasped is present.

As mentioned above, hand piece 110 may include components that are not capable of being autoclaved. Thus, oral piece 102 may be detached from hand piece 110 so that it can be autoclaved. Hand piece 110 includes a connector 113, and handle 112. Connector 113 is adapted to couple with connector 105 of oral piece 102. The coupling may be electrical. For example, connector 113 may supply power to oral piece 102 through connector 105. In addition, hand piece 110 and oral piece 102 may transmit and receive data from one another via connectors 113 and 105.

Handle 112 enables a health care provider to grasp x-ray sensor device 100. Like appendage 104, handle 112 may have an elongated shape, such as a cylinder, and may be hollow on the inside to conceal electronic components. For example, handle 112 of hand piece 110 may conceal a power supply 926, a processor 916, and accelerometer 920 and/or gyroscope 922 (as illustrated in FIG. 9). On handle 112 is button 114, which accepts input from the health care provider. In some aspects, handle 112 is completely covered by appendage 104, so a health care provider does not literally grasp it when x-ray sensor device 100 is in use.

Determining the Position of an x-Ray Sensor with an Inertial Measurement Unit

As discussed earlier, with respect to FIG. 1A, an x-ray sensor head 103 that includes one or more cameras 101 and an inertial measurement unit 902 to enable the calculation of the position (location and/or orientation) of x-ray sensor head 103 in a patient's mouth. In some aspects, the position of an x-ray sensor head 103 in a patient's mouth is determined through dead reckoning, that is, a repeating process where at each step a current position is determined by accumulating the change from a previous determined position. Since this method uses accumulation, it is known to also accumulate errors, thus the accuracy may be acceptable for a short period but may degrade with time and the determined position may become too erroneous in prolonged periods of time. To reduce the errors, in some aspects, a Kalman filter is used to fuse inputs from the inertial measurement unit 902 and inputs from the camera(s). By continuously analyzing images captured at a camera, values representing an estimation of motion may be determined. This estimation may be done, for example, by observing time successive images and identifying the shift in positions of features that are present in all said images. The shift and the time between capture of these images is used to estimate a motion of the camera. A Kalman filter combines values generated from the estimation of camera motion and values generated from the output of the inertial measurement unit 902, to determine a more accurate estimation of the position of x-ray sensor head 103 inside the patient's mouth.

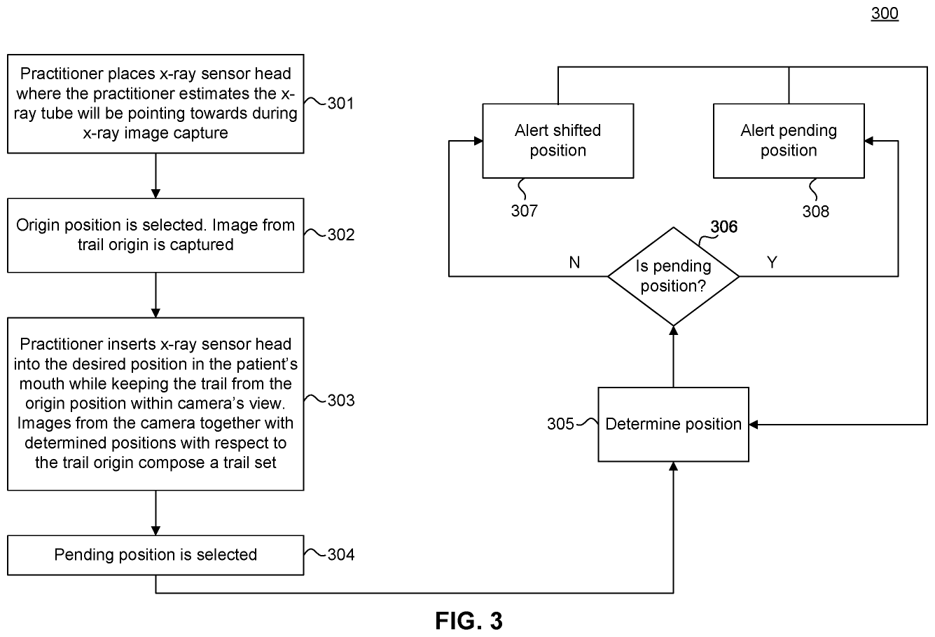

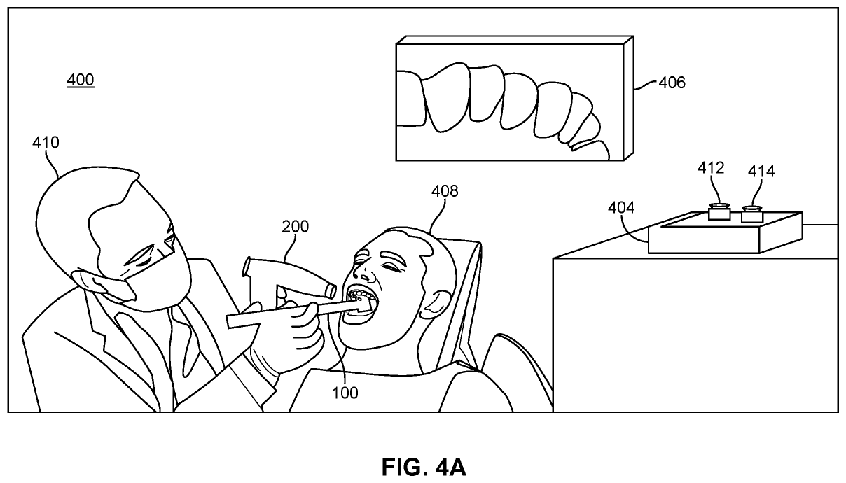

FIG. 3 is a flowchart illustrating an example method 300 of determining a position of an x-ray sensor head 103 (shown in FIG. 1A) in a patient's mouth in respect to one or more images of the patient's face. Method 300 starts at step 301, where a practitioner that wishes to capture an x-ray image brings x-ray sensor head 103 closer to a patient's face, approximately parallel to the area of the face where the practitioner estimates that x-ray tube 200 will be pointing towards during capture of the x-ray image, so that camera 101 is observing that arca.

At step 302, an origin position of a trail is selected and thereafter changes of the position of the x-ray sensor head 103 are determined with reference to this origin. There are several ways to trigger the selection of a trail origin. In some aspects, the practitioner may trigger the selection of a trail origin, for example by pressing a button, or using a voice command. In some aspects, the selection of a trail origin is triggered automatically, for example, by analyzing the images captured at a camera 101 and determining that there is no motion for a threshold period of time and the image has pixels which consist of colors found in the human faces, and in a quantity above some thresholds, or, for example, by feeding the images into a machine learning inference that has a model trained to identify the parts of the human face relevant for x-ray capture for dental health purposes, and choose to trigger based on one or more of the inference results. In some aspects, the practitioner receives a notification that a trail origin was selected, for example, an indication light is turned on. The image captured at the trail origin position is added to a set of trail images recorded for later use.

At step 303, the practitioner shifts the position of x-ray sensor head 103 from the trail origin into the desired position inside the patient's mouth. The practitioner does so while keeping a camera 101 continuously observing the patient's tissue in a manner that allows a Kalman filter to continuously determine the position of x-ray sensor head 103 using values generated from the estimation of camera 101 motion and values generated from the output of the inertial measurement unit 902, virtually recording a trail starting at the trail origin. The recording of a trail produces a set of images captured by camera 101 together with their positions with respect to the trail origin position. The set of images are hereafter referred to as a trail set. In some aspects, the trail recorded includes images from the face of the patient, and images from the mouth of the patient are not included in the set of images of the trail. Images may be recognized as captured inside the patient's mouth by feeding the images into a machine learning inference that has a model trained to classify images inside a human's mouth from images not inside a mouth.

At step 304, x-ray sensor head 103 arrives at a desired position for the x-ray image to be captured. The position is designated as the pending position, where the x-ray sensor head 103 is pending for x-ray radiation in order to capture an x-ray image. A pending position defines a position of x-ray sensor head 103 with reference to an origin position. There are several ways to trigger the selection of a pending position. In some aspects, it is the practitioner that triggers the selection of a pending position, for example by pressing a button, or using a voice command. In some aspects, the selection of pending position is triggered automatically, for example, determining that x-ray sensor head 103 is in a constant position for some period of time larger than a threshold, or, for example, by feeding the images into a machine learning inference that has a model trained to identify images that indicate reaching a pending position, and choose to trigger based on one or more of the inference results. In some aspects, the practitioner receives a notification that the pending position was selected.

At step 305, the current position of x-ray sensor head 103 is determined. The current position is determined with reference to the origin position determined at step 303. In some aspects, the current position is determined using output from a Kalman filter that may continuously determine the position of x-ray sensor head 103 using values generated from the estimation of camera 101 motion and values generated from the output of the inertial measurement unit 902.

At step 306, it is checked whether the x-ray sensor head 103 maintained the pending position or not. In order to determine whether the x-ray sensor head 103 maintained the pending position, the pending position, determined at step 304, is compared to the current position of x-ray sensor head 103 determined at step 305 in order to assert if they are substantially equal, or equivalently by determining a shift from the pending position, for example, by comparing images captured at the pending position and current position and identifying the shift in positions of features that are present in both images to determine if the sensor has substantially maintained its position.

Depending on the result, a notification that the x-ray sensor head 103 has maintained its position at the pending position, or has shifted from that position may be presented to the practitioner at steps 307 or 308, and the method cycles back to step 305.

Systems and Methods that Utilize the Digital x-Ray Sensor

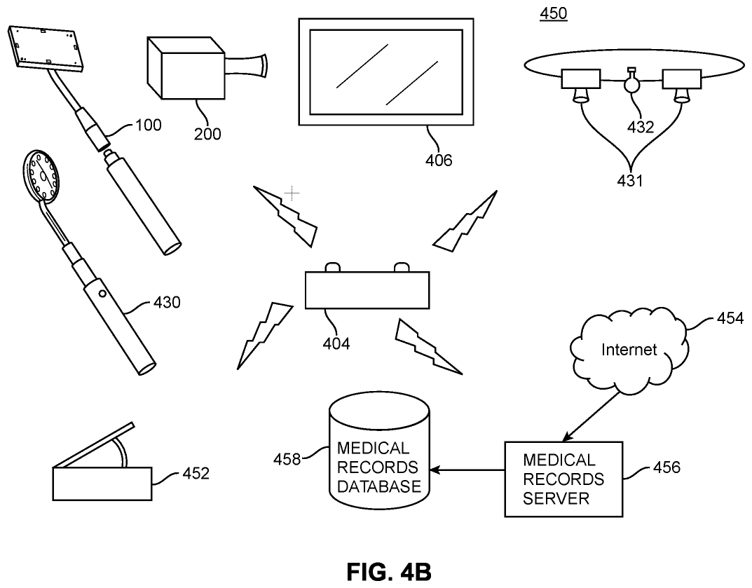

FIG. 4A illustrates an embodiment of room 400 of a dental office including a system for displaying video and images as viewed by one or more cameras integrated into an x-ray sensor. Room 400 shows a practitioner 410 capturing an x-ray image showing teeth of patient 408 using x-ray sensor device 100 and a handheld x-ray tube 200. Display 406 shows what is visible to one or more cameras 101 in x-ray sensor device 100. While positioning x-ray sensor head 103 in patient's 408 mouth, practitioner 410 may watch the intraoral space in display 406, thus placing x-ray sensor head 103 in its desired position with ease and accuracy. Practitioner 410 may additionally request to capture an image or a video, for example by a voice command.

Once x-ray sensor head 103 is in the desired position, practitioner 410 brings x-ray tube 200 to proximity with patient's 408 face, pointing the x-ray emitting end of x-ray tube 200 towards x-ray sensor head 103 which is located inside patient's 408 mouth. In the depicted embodiment, x-ray tube 200 is a handheld tube, and practitioner 410 holds it in one hand, while holding x-ray sensor device 100 in the other hand. A practitioner may align x-ray sensor head 103 and x-ray tube 200 as the grasp of handle 112 provides a tactile attachment to the x-ray sensor device 100 which may allow for a practitioner to sense an estimate of the required position of x-ray tube 200 so that it aligns with x-ray sensor head 103. Aligning the x-ray sensor head 103 and x-ray tube 200 is further described below.

When x-ray tube 200 is in alignment with x-ray sensor head 103, an x-ray image is captured. Thereafter the captured x-ray image may be shown in display 406. Capturing an x-ray image may be triggered by a practitioner 410, for example by pressing a button on x-ray tube 200 or by voice command.

Also inside room 400 is a base station 404. Base station 404 includes cradles 412 and 414. Base station 404 allows multiple handles 112 to dock.

Once healthcare practitioner 410 is no longer using x-ray sensor device 100, he may place handle 112 of x-ray sensor device 100, on one of cradles 412 and 414. When handle 112 is docked with cradles 412 or 414, base station 404 may charge handle 112.

Base station 404 also provides for communication with x-ray sensor device 100. In particular, x-ray sensor device 100 transmits images from its cameras 101 or from its x-ray capturing element 210, sound captured at its microphone, measurements by inertial measurement unit 902, or perhaps determined position from a Kalman filter, as well as other information to base station 404. In some aspects, the x-ray sensor device 100 may transmit the information through processor 904 or processor 916 (as illustrated in FIG. 9). Base station 404 may transmit information for presentation on display 406. Embodiments of display 406 include a wall mounted screen, tablet, smartphone, a watch or perhaps a display integrated into x-ray tube 200. Base station 404 may also provide communication with x-ray tube 200. For example, to trigger an x-ray image capture, or to display images from camera 101 or captured x-ray images on a display mounted or integrated to x-ray tube 200, or perhaps for x-ray tube 200 to inform base station 404 that x-ray radiation emission is in progress. The communication paths are illustrated in FIG. 4B.

FIG. 4B is an architecture diagram 450 of the system in FIG. 4A. In addition to the components in FIG. 4A, architecture diagram 450 illustrates a smart mirror device 430, which is described in U.S. Pat. No. 10,188,278 and incorporated herein, one or more room cameras 431, a room microphone 432, a foot pedal 452, a medical records server 456, and a medical records database 458.

X-ray sensor device 100, x-ray tube 200, room microphone 432, room cameras 431, display 406, and foot pedal 452 may be connected using a wireless connection, such as Wi-Fi. In particular, base station 404 may act as a Wi-Fi router and provide network routing and address information to x-ray sensor device 100, x-ray tube 200, room microphone 432, room cameras 431, display 406, and foot pedal 452.

Foot pedal 452 provides a way for the practitioner to input information in a hands-free manner. For example, to request the capturing of an x-ray image.

Room cameras 431 are positioned so they are able to capture the patient's face during treatment, and possibly the area surrounding the face. For example, room cameras 431 may be mounted on the dental lamp that a dentist or other practitioners use to aim towards a patient's mouth to illuminate, or perhaps attached to the ceiling above the dental chair. In some embodiments, a pair of room cameras 431 are configured to provide stereoscopic images. The images may be used to determine relative position between objects that appear in the images during a dental procedure, for example, an x-ray tube 200 and an x-ray sensor head 103.

Room microphone 432 captures sound in the room during a patient's treatment. In some embodiments, the sound is processed to enable a practitioner to control the system through voice commands.

Base station 404 is connected to medical records server 456 via one or more networks 454, such as the Internet. Base station 404 may be connected to the Internet either through a wireless or wired LAN in the dental office. Medical records server 456 is a computerized process adapted to run in one or more remote server computers. Medical records server 456 may, for example, be a cloud server. Medical records server 456 is further connected to an archival medical records database 458. Medical records database 458 stores medical record information, including x-ray images collected from x-ray sensor device 100.

Capturing a Panoramic x-Ray Using an x-Ray Sensor

As mentioned with respect to FIG. 4A, images from a camera 101 of the x-ray sensor head 103 are captured and transmitted to base station 404 while an x-ray sensor head 103 is inside the patient's 408 mouth. A practitioner 410 may now proceed to capture a panoramic x-ray image, which is a set of x-ray images stitched together to produce a larger x-ray image covering all of the patient's teeth, or perhaps a contiguous section of the teeth.

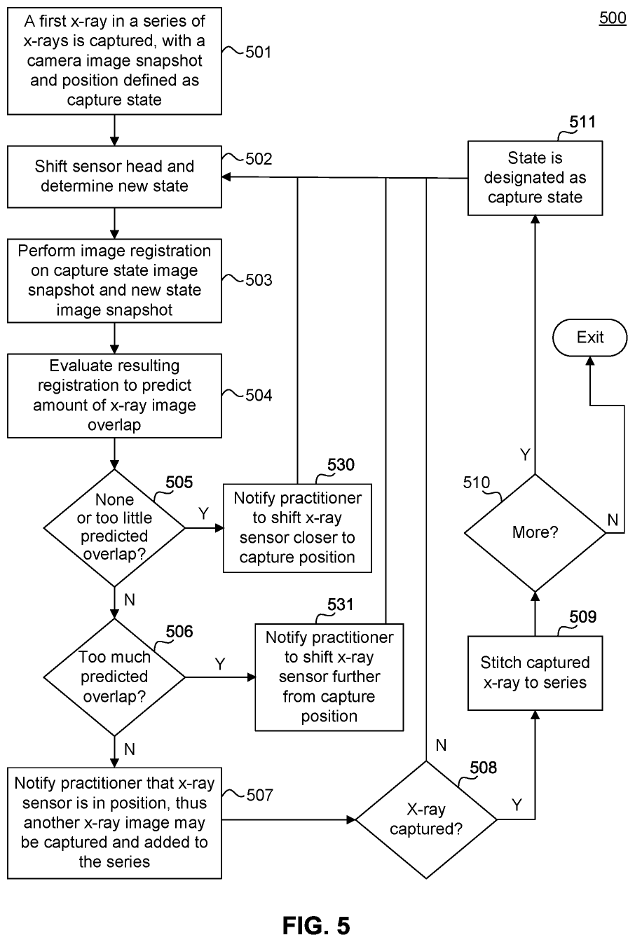

FIG. 5 is a flowchart illustrating an example method 500 for capturing a series of x-ray images to form a set for generating a panoramic x-ray, using an x-ray sensor device 100 with an x-ray sensor head 103 having an integrated camera. Method 500 starts at step 501, where an x-ray image is captured, and a state of x-ray sensor head 103 is determined, the state of x-ray capturing includes an image snapshot captured by camera 101 at the time of x-ray image capture, and may also include the position of x-ray sensor head 103 at the same time. The state determined at this time is designated as the capture state, the state at which an x-ray image was captured. The capture state may further include features extracted from the image snapshot, for example using a feature detection algorithm such as SIFT, SURF or others.

In step 502, practitioner 410 shifts the x-ray sensor head 103 to a new position seeking the next position for capturing the next x-ray image in the series, and a new state of x-ray sensor head 103 is determined, including a new image snapshot, and possibly position or features.

In step 503, the image snapshot recorded in the capture state and the image snapshot recorded in the new state are registered, in order to produce a single integrated image and/or determine if a single image may be generated using the two snapshots.

In step 504, the result of the registration is evaluated to predict the amount of overlap between the x-ray image of the capture state, and an x-ray image that would have been captured in the new state. The prediction may use an indication that the registration of the image snapshot at both states is able to produce a single smooth integrated image out of the two, the number and/or locations of the shared features among the snapshots, the difference in position, including the spatial shift and difference in orientation between the two states, the results received from a machine learning inference using a model trained to input at least the two states and estimate the probability for success of stitching an x-ray images that may be captured at the new position, with a previously captured x-ray image.

In step 505, it is determined if the predicted amount of overlap is too small or not existing for an x-ray captured at that position to be able to stitch with the x-ray images set. If this is the case, the practitioner 410 is notified, in step 530, in some aspects with an indication light, to shift the x-ray sensor head 103 closer to the position of the capture state, and the process repeats from step 502.

If the predicted overlap is not too small, it is determined in step 506 whether the predicted overlap suggests there might be too much overlap. Although more overlap is better for stitching algorithms, the goal is to expose patient 408 to less radiation, thus capturing the minimum amount of x-ray images that will produce the desired panoramic x-ray. In some aspects, an overlap amount of 10-15% may suffice. If the predicted overlap is too much, the practitioner 410 is notified, in step 531, in some aspects with an indication light, to shift the x-ray sensor head 103 further from the position of the capture state, and the process repeats from step 502.

In step 507, the predicted overlap is not too small nor too much, and the practitioner 410 is notified, perhaps with an indication light, that an x-ray image may be captured at that position.

A practitioner 410 may trigger the capturing of an x-ray, asynchrously to steps 502 to 507. In step 508, it is checked whether an x-ray was captured at a new state. If the x-ray is not captured at the new state, the process repeats from step 502 to provide continuous feedback to practitioner 410.

In step 509, when an x-ray image is captured at the position of the new state, the image is stitched together with previously captured x-ray images of the series of images forming at least a section of the desired panoramic x-ray. In some aspects, the image is transmitted to a display for presentation. In some aspects, the image is stitched together with previously captured images of this series to form one integrated image.

In step 510, if practitioner 410 is satisfied that the series is completed, the process exits.

In step 511, in preparation for the next cycle, the new state is now designated as the capture state, replacing the previous designation, and the process repeats with this new designation to step 502 for assisting with capturing another x-ray image in the series.

Systems and Methods for Aligning an x-Ray Tube and an x-Ray Sensor

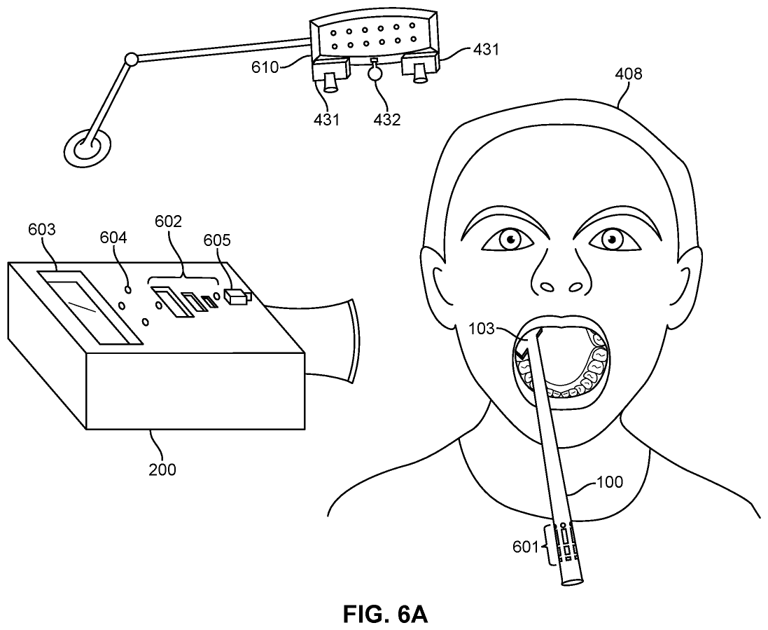

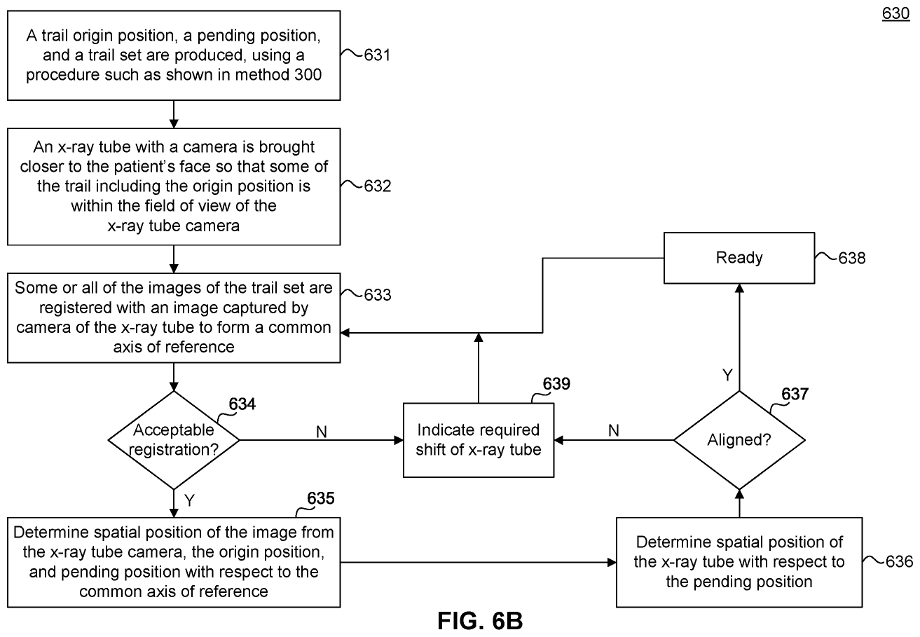

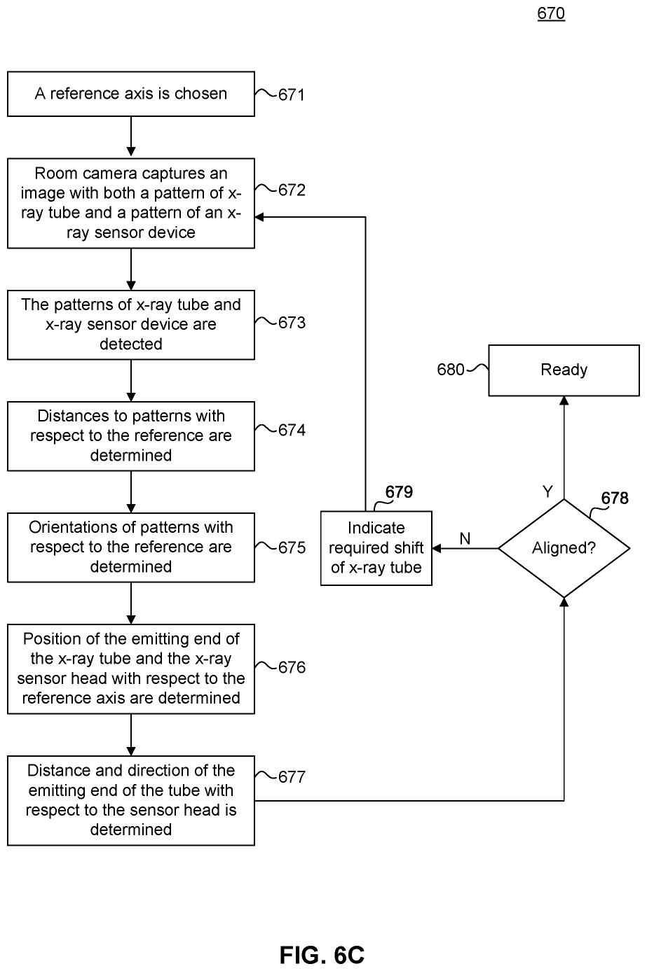

FIG. 6A depicts an embodiment of some of the components described with respect to FIGS. 4A and 4B, and specifically, an x-ray sensor device 100, an x-ray tube 200, room cameras 431, and room microphone 432. Additionally, depicted room cameras 431 and room microphone 432 are attached to a dental lamp 610.

X-ray tube 200, has a display 603, light indicators 604, pattern 602 and a camera 605. Additionally, x-ray tube 200 may also have an inertial measurement unit. Display 603 may present an x-ray image that was captured by x-ray sensor device 100. Indications and status may also be shown on display 603. Light indicators 604 may indicate instructions to a practitioner, such as, for example, shift the x-ray tube 200 leftwards or rightwards. In some aspects, light indicators 604 may indicate a status that x-ray tube 200 is correctly aligned to x-ray sensor head 103.

Pattern 602 provides physical details for identifying x-ray tube 200 and determining its position using one or more images from a camera, and particularly room cameras 431. X-ray tube pattern 602 may be colored, engraved, or perhaps raised from the surface of x-ray tube 200.

Camera 605 captures images that may include at least a portion of the area toward which the emitting end of x-ray tube 200 will emit x-ray radiation. Images from camera 605 and data from x-ray tube 200 and inertial measurement unit 902 may be fused by a Kalman filter for determining a position of x-ray tube 200 with improved accuracy.

In the depicted aspect, x-ray sensor device 100 is located inside the mouth of patient 408, and hand piece 110 is visible outside the mouth. Similarly to pattern 602 of x-ray tube 200, pattern 601 of x-ray sensor device 100 may be present in images captured by room cameras 431.

Dental lamp 610 may be mounted or have an arm that enables it to move. One or more room cameras 431 may be mounted or integrated into the lamp 610. In some aspects, a room camera 431 has both x-ray tube 200 and x-ray sensor device 100 in its field of view, and more specifically, pattern 601 of x-ray sensor device 100 and pattern 602 of x-ray tube 200, thus images captured by room cameras 431 may contain both.

The configuration embodied in FIG. 6A illustrates method 300 for determining a position of an x-ray sensor head 103 in a patient's mouth in reference to one or more images of the patient's face. FIG. 6B is a flowchart illustrating an example method 630 for positioning an x-ray tube 200 towards a patient's 408 face so that it is aligned with an x-ray sensor head 103, using a camera 605 of an x-ray tube 200 and one or more images of the patient's face such that their positions with respect to an x-ray sensor head 103 are determined, for example, by a method 300.

Method 630 starts at step 631, where a trail origin position, a pending position, and a trail set are generated, for example by using method 300, as described with respect to FIG. 3.

At step 632, x-ray tube 200 is pointing towards the face of the patient, aiming towards an area which was captured in images in the trail set.

At step 633, registration of at least some of the images from the set of trail images with an image from camera 605 produces spatial correlation to determine a common spatial axis of reference.

At step 634, the result of the registration is evaluated. If determined that the registration is not acceptable, for example due to low correlation of the images and/or because the emitting end of x-ray tube 200, and thus also camera 605, are not pointing towards the face where the trail images were captured, in step 639 an indication to shift x-ray tube 200 is provided in display 603 or light indicators 604. The cycle continues repeating step 633.

If the evaluation of registration of step 634 is acceptable, in step 635 the position of the image from camera 605, the image captured at the trail origin position, and the image captured at the pending position are determined with respect to a common axis.

In step 636, the spatial position of the x-ray tube 200 is calculated with respect to the pending position of the x-ray sensor head 103.

In step 637, the relative position calculated in step 636 is evaluated to determine if x-ray tube 200 is perpendicularly aligned towards the center the x-ray sensor head 103, or at least considered aligned within a threshold.

If x-ray tube 200 is determined to be aligned, in step 638, an indication is presented to inform a practitioner to keep the x-ray tube 200 in the position for capturing an x-ray image. The process repeats with step 633 so that alignment is continuously verified.

If the x-ray tube 200 is determined to not be aligned, in step 639, an indication that x-ray tube 200 is shifting is provided. The indication may also include a direction that the x-ray tube 200 is shifting. The cycle continues repeating step 633.

FIG. 6C is a flowchart illustrating an example method 670 for positioning an x-ray tube 200 towards the face of patient 408 so that it is aligned with an x-ray sensor head 103 using a room camera 431.

Method 670 starts at step 671, where a reference axis is selected. A reference axis defines a center and axes, oftentimes orthogonal axes, in 3 directions that may be used to describe measurements, such as length or angle in a space. The reference axis serves as an anchor for the following steps and allows for consistent calculations. An example selection may be, for example, an axis origin that is at the center of one camera and one of the axes points directly towards the center of the camera's field of view. In another aspect, the origin may be at a center point between multiple cameras.

At step 672, a room camera 431 captures an image where both a pattern 602 of x-ray tube 200 and a pattern 601 of x-ray sensor device 100 are present in the image.

At step 673, the patterns 601 and 602 are detected within the image captured by room cameras 431. Detection of the patterns can be done using a machine learning inference with a model trained to detect pattern 601 of the x-ray sensor device 100 and pattern 602 of the x-ray tube 200, for example using an R-CNN model or a YOLO model.

At step 674, a distance to a pattern 601 and to pattern 602 are determined with respect to the reference axis. In some aspects, a distance is determined in relation to the size of the pattern in an image from one or more room cameras 431. In this aspect, the larger the size of the pattern, the closer a pattern is to the camera and in relation to the location of a detected pattern within the image. In some aspects, two or more room cameras 431 are present and stereoscopic images are captured. A distance to a pattern 601 or 602 may be determined using depth information calculated by measuring differences between the detected patterns 601 and 602 as appearing in images captured by room cameras 431 while observing the patterns at different angles.

At step 675 the spatial orientation of pattern 601 and pattern 602 is determined with respect to the reference axis. The spatial orientation refers to the orientation of pattern 601 and pattern 602 as they are captured by room cameras 431. The spatial orientation provides further information with respect to the position of the x-ray sensor device 100 and the x-ray tube 200.

At step 676 the position of the emitting end of x-ray tube 200 and the position of x-ray sensor head 103 is determined with respect to the reference axis. The position of the emitting end of x-ray tube 200 is determined using the position of pattern 602 and a transformation appropriate for the physical shape of x-ray tube 200. The position of x-ray sensor head 103 is determined using the position of pattern 601 and a transformation appropriate for the physical shape of x-ray sensor device 100 and the angle of rotation or amount of linear displacement of x-ray sensor head 103 with respect to appendage 104 of x-ray sensor device 100 as respectively measured from rotating element 118 or slider element 119 of FIGS. 1C-1D.

At step 677 the distance and direction of the emitting end of x-ray tube 200 with respect to the position of the center of x-ray sensor head 103 is determined.

At step 678, the distance and direction calculated in step 677 are evaluated to determine if x-ray tube 200 is perpendicularly aligned towards the center the x-ray sensor head 103, or determined to be aligned within a threshold.

If x-ray tube 200 is determined to be aligned, at step 680, a ready indication is presented to inform a practitioner to keep x-ray tube 200 in the position for capturing an x-ray image. The process repeats with step 672 so that alignment is continuously verified.

If x-ray tube 200 is determined to not be aligned, at step 679, an indication that x-ray tube 200 is shifting is provided. The indication may also include a direction that the x-ray tube 200 is shifting. The cycle continues repeating at step 672.

### Alternative Embodiments of an x-Ray Sensor Device

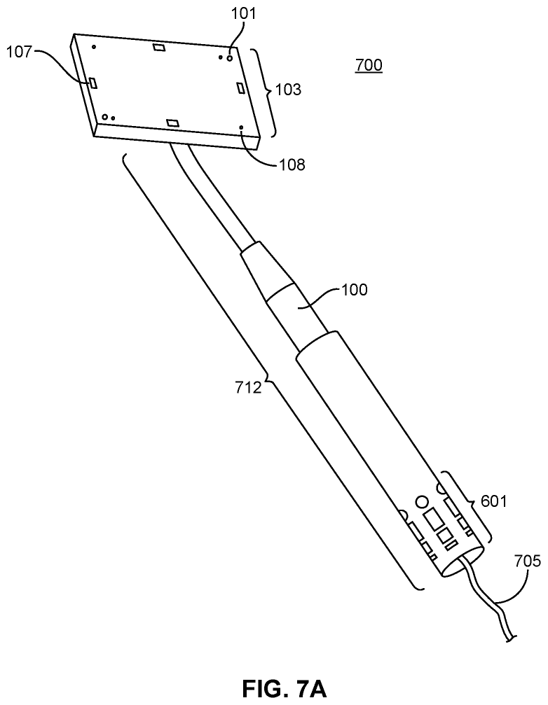

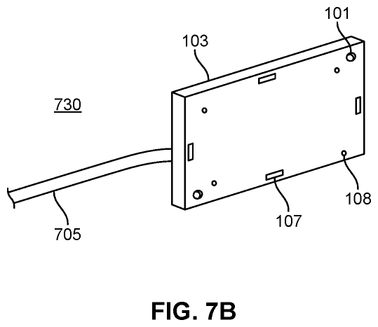

FIG. 7A is an embodiment of an alternative configuration 700 of x-ray sensor device 100 where oral piece 102 is combined with hand piece 110, with a handle 712, where appendage 104 is combined with handle 112 of configuration 100 of FIG. 1. Alternative configuration 700 also depicts x-ray sensor head 103, pattern 601 and cable 705. Handle 712 may be hollow. X-ray sensor head 103 may be connected to a base station 404 (not shown) through cable 705 that extends through handle 712.

Cable 705 may electronically and physically connect x-ray sensor head 103 with a base station 404, for example, cable 705 may be a USB cable. X-ray sensor head 103 may receive and send control and data signals and connect to power base station 404 through cable 705.

FIG. 7B is an embodiment of an alternative configuration 730 of x-ray sensor device 100 where x-ray sensor head 103 may be connected to a base station 404 (not shown) through cable 705.

Device with Lights and Method for Aligning an x-Ray Tube and x-Ray Sensor

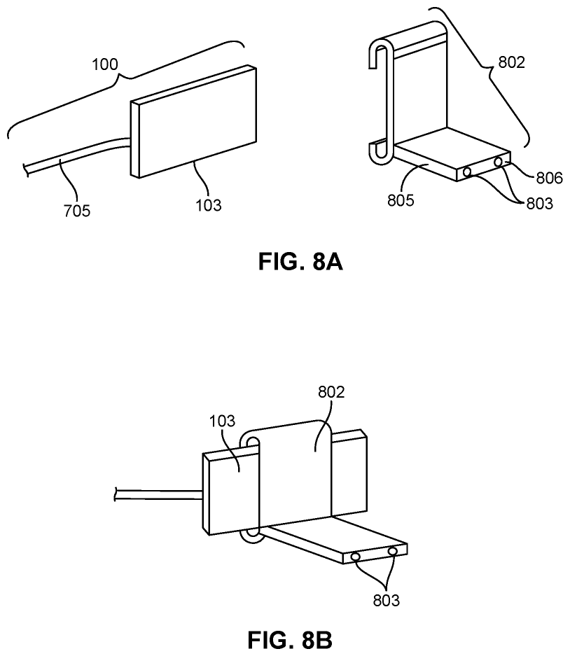

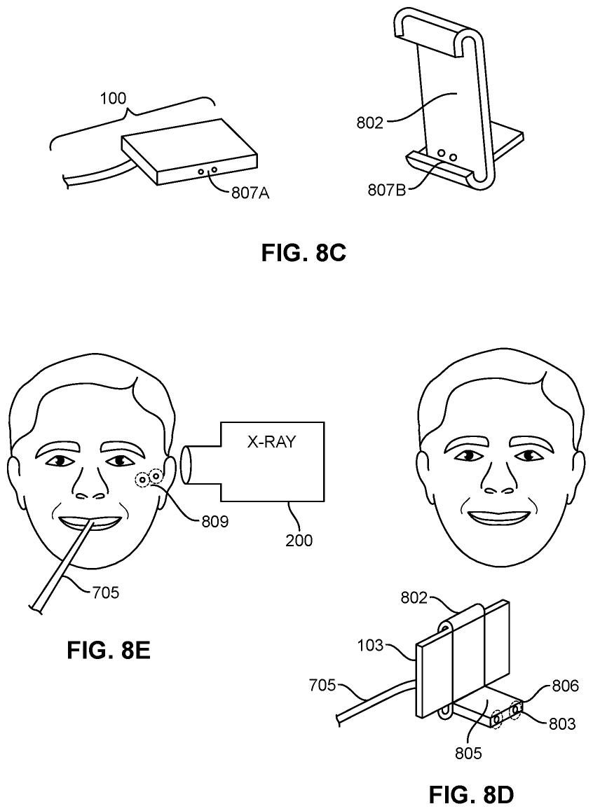

FIGS. 8A-C are diagrams illustrating an x-ray sensor device and a bitewing holder with lights capable of attaching to the x-ray sensor device, according to an aspect.

FIG. 8A depicts an aspect of an x-ray sensor device 100 and a bitewing holder 802 when detached, while FIG. 8B shows an embodiment when x-ray sensor device 100 and a bitewing holder 802 are attached. X-ray sensor device 100 has an x-ray sensor head 103 and a cable 705. X-ray sensor head 103 may physically and/or electronically connect to a base station 404 (not shown) through cable 705 to receive and send control and data signals and to receive power. Bitewing holder 802 has LEDs 803 at a tip 806 of its bitewing 805. Bitewing 805 is an extension from bitewing holder 802 for a patient to bite, for keeping bitewing holder 802, and consequently x-ray sensor head 103, in a fixed position while an x-ray image is captured. FIG. 8C depicts an embodiment of x-ray sensor device 100 and of bitewing holder 802 from a different perspective than shown in FIGS. 8A and 8B, where electronic connections 807A and 807B are shown. To power and control LEDs 803, electronic connection 807B of bitewing holder 802 electronically couples to electronic connection 807A of x-ray sensor device 100.

FIGS. 8D and 8E are diagrams illustrating two steps in a process for alignment of an x-ray tube head to an x-ray sensor head inside a patient's mouth, according to an aspect. FIG. 8D depicts an embodiment of x-ray sensor head 103 attached to bitewing holder 802 while being prepared to be inserted into a patient's mouth in an orientation desired by the practitioner, in the example, x-ray sensor head 103 of x-ray sensor device 100 is laid above bitewing 805 of bitewing holder 802. In FIG. 8E, x-ray sensor head 103 together with bitewing holder 802 are positioned inside the patient's mouth, while the patient bites on bitewing 805, and tip 806 of bitewing 805 is in proximity of the patient tissue, in this example the patient check (components inside the mouth are not shown). Cable 705 exits the patient mouth and is connected to a base station 404 (not shown), while LEDs 803 are emitting light. The light emitted from LEDs 803 has enough intensity to traverse the patient's tissue, so it is presently viewed from the outside as light marker 809. The light marker 809 indicates to the practitioner the location of the bitewing tip 806 inside the patient's mouth. Considering the position of the sensor relative to the bitewing tip 806, which is determined by the practitioner prior insertion into the patient's mouth, the practitioner can determine where x-ray sensor head 103 is located, thus where to aim x-ray tube head 200. For example, if x-ray sensor head 103 was positioned above the bitewing 805, the practitioner should aim above light marker 809, in order for the x-ray radiation emitted from x-ray tube head 200 to be directed to x-ray sensor head 103 located inside the mouth.

In a plurality of aspects, one or more of LEDs 803 may exist. In embodiments, at least some of LEDs 803 are not located at the tip of the bitewing, but elsewhere, for example on the lower end of bitewing holder 802, or at the back area of bitewing holder 802, to be attached at the back of x-ray sensor head 103. The light from LEDs 803 may be transferred, for example using a light pipe or a fiber optic, to the tip of bitewing holder 802 to achieve proximity to the inner cheek. This is done despite the loss of light intensity in such a configuration in order to cause lower temperatures in the areas touching the human tissue, or for reducing obstruction to the x-ray radiation from arriving at the x-ray sensor. In some aspects, the x-ray sensor head 103 and bitewing holder 802 are combined into one and do not detach from each other.

Estimation terms, such as “approximate,” “approximately,” “about,” and the like may be used herein to indicate the value of a given quantity that may vary based on a particular technology and/or certain parameter(s). For example, the estimation term may modify amounts, sizes, formulations, parameters, and other quantities and characteristics, and need not be exact, but may be approximate and/or larger or smaller, as desired, reflecting tolerances, conversion factors, rounding off, measurement error and the like, and other factors known to those of skill in the art. Such estimation terms may indicate a value of a given quantity that varies within, for example, 0-10% of the value (e.g., +0.5%, +5%, or +10% of the value).

Directional and spatially relative terms, such as “inner,” “outer,” “proximal,” “distal” and the like, may be used herein for ease of description to describe one element or feature's relationship to another element(s) or feature(s) as illustrated in the figures. The directional and spatially relative terms are intended to encompass different orientations of the device in use or operation in addition to the orientation depicted in the figures. The apparatus may be otherwise oriented (rotated 90 degrees or at other orientations) and the directional and spatially relative descriptors used herein may likewise be interpreted accordingly.

As used herein, the singular forms “a,” “an” and “the” include plural referents unless the context clearly dictates otherwise. Thus, for example, reference to “a” component includes aspects having two or more such components, unless the context clearly indicates otherwise.

It is to be understood that the phraseology or terminology herein is for the purpose of description and not of limitation such that the terminology or phraseology of the present specification is to be interpreted by those skilled in relevant art(s) in light of the teachings herein.

The aspect(s) described, and references in the specification to “one aspect,” “an aspect,” “an example aspect” and the like indicate that the aspect(s) described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to effect such feature, structure, or characteristic in connection with other aspects whether or not explicitly described.

It will be apparent to those skilled in the art that various modifications and variations can be made to the aspects described herein without departing from the spirit and scope of the claimed subject matter. Thus it is intended that the specification cover the modifications and variations of the various aspects described herein provided such modification and variations come within the scope of the appended claims and their equivalents.

What is claimed is:

1. A method for aligning a dental instrument comprising:
capturing a first image from a first image sensor affixed to an x-ray sensor head, the first image captured at an origin position;
determining a pending position of the x-ray sensor head with reference to the origin position;
determining whether the first image shares a common axis of reference with an x-ray tube based on a correlation between the first image and a second image captured by a second image sensor affixed to the x-ray tube;
calculating a position of the x-ray tube with reference to the pending position of the x-ray sensor head and the common axis of reference; and
based on the position of the x-ray tube, determining whether the x-ray tube is perpendicularly aligned towards the x-ray sensor head when at the pending position.

2. The method of claim 1, wherein the pending position represents a desired position for an x-ray image of a patient's mouth to be captured, further comprising:
capturing, by the first image sensor, a trail set comprising a plurality of images continuously captured by the first image sensor as the x-ray sensor head is shifted from the origin position representing a starting point of the trail set to the pending position;
calculating a respective position for each image in the plurality of images based on the origin position; and
for a respective image from the trail set, determining whether the respective image shares a common axis of reference with the x-ray tube based on a correlation between the respective image and a third image captured by the second image sensor.

3. The method of claim 1, wherein the x-ray tube comprises a first inertial measurement unit, and wherein the x-ray sensor head comprises a second inertial measurement unit, further comprising:
detecting, by the first inertial measurement unit, movement of the x-ray tube;
detecting, by the second inertial measurement unit, movement of the x-ray sensor head;
based on the detected movement of the x-ray tube, calculating an orientation of the x-ray tube; and
based on the detected movement of the x-ray sensor head, calculating an orientation of the x-ray sensor head, wherein the determining whether the x-ray tube is perpendicularly aligned occurs based on the calculated orientation of the x-ray tube and the x-ray sensor head.

4. The method of claim 1, wherein the determining whether the x-ray tube is perpendicularly aligned towards the x-ray sensor head further comprises:
capturing, by a third image sensor, a room image, wherein the room image comprises a first marker located on the x-ray tube and a second marker located on the x-ray sensor head; and
based on a distance between the first and second markers, determining whether the x-ray tube is perpendicularly aligned towards the x-ray sensor head.

5. The method of claim 1, wherein the x-ray sensor head further comprises a light that illuminates a patient's mouth such that the light is visible through a patient's cheek, wherein the light indicates the location of the x-ray sensor head inside the patient's mouth.

6. The method of claim 1, further comprising:
when the x-ray tube is aligned with the x-ray sensor head, notifying a healthcare practitioner, by an application, that the x-ray tube and the x-ray sensor head are aligned.

7. The method of claim 6, further comprising:
when the x-ray tube is not aligned with the x-ray sensor, notifying the healthcare practitioner, by the application, that the x-ray tube requires shifting.

8. The method of claim 1, further comprising:
when the x-ray tube is aligned with the x-ray sensor head, capturing a first x-ray image by an x-ray capturing element affixed to the x-ray sensor head.

9. The method of claim 1, wherein the pending position of the x-ray sensor head is determined to occur when the x-ray sensor head is in a constant position for a period of time larger than a threshold.

10. The method of claim 1, wherein the x-ray sensor head is attached to an appendage comprising a display.

11. A non-transitory computer-readable device having instructions stored thereon that, when executed by at least one computing device, cause the at least one computing device to perform operations comprising:
capturing a first image from a first image sensor affixed to an x-ray sensor head, the first image captured at an origin position;
determining a pending position of the x-ray sensor head with reference to the origin position;
determining whether the first image shares a common axis of reference with an x-ray tube based on a correlation between the first image and a second image captured by a second image sensor affixed to the x-ray tube;
calculating a position of the x-ray tube with reference to the pending position of the x-ray sensor head and the common axis of reference; and
based on the position of the x-ray tube, determining whether the x-ray tube is perpendicularly aligned towards the x-ray sensor head when at the pending position.

12. The non-transitory computer-readable device of claim 11, wherein the pending position represents a desired position for an x-ray image of a patient's mouth to be captured, further comprising:
capturing, by the first image sensor, a trail set comprising a plurality of images continuously captured by the first image sensor as the x-ray sensor head is shifted from the origin position representing a starting point of the trail set to the pending position;
calculating a respective position for each image in the plurality of images based on the origin position; and
for a respective image from the trail set, determining whether the respective image shares a common axis of reference with the x-ray tube based on a correlation between the respective image and a third image captured by the second image sensor.

13. The non-transitory computer-readable device of claim 11, wherein the x-ray tube comprises a first inertial measurement unit, and wherein the x-ray sensor head comprises a second inertial measurement unit, further comprising:
detecting, by the first inertial measurement unit, movement of the x-ray tube;
detecting, by the second inertial measurement unit, movement of the x-ray sensor head;
based on the detected movement of the x-ray tube, calculating an orientation of the x-ray tube; and
based on the detected movement of the x-ray sensor head, calculating an orientation of the x-ray sensor head, wherein the determining whether the x-ray tube is perpendicularly aligned occurs based on the calculated orientation of the x-ray tube and the x-ray sensor head.

14. The non-transitory computer-readable device of claim 11, wherein the determining whether the x-ray tube is perpendicularly aligned towards the x-ray sensor head further comprises:
capturing, by a third image sensor, a room image, wherein the room image comprises a first marker located on the x-ray tube and a second marker located on the x-ray sensor head; and
based on a distance between the first and second markers, determining whether the x-ray tube is perpendicularly aligned towards the x-ray sensor head.

15. The non-transitory computer-readable device of claim 11, wherein the x-ray sensor head further comprises a light that illuminates a patient's mouth such that the light is visible through a patient's cheek, wherein the light indicates the location of the x-ray sensor head inside the patient's mouth.

16. The non-transitory computer-readable device of claim 11, further comprising:
when the x-ray tube is aligned with the x-ray sensor head, notifying a healthcare practitioner, by an application, that the x-ray tube and the x-ray sensor head are aligned.

17. The non-transitory computer-readable device of claim 16, further comprising:
when the x-ray tube is not aligned with the x-ray sensor, notifying the healthcare practitioner, by the application, that the x-ray tube requires shifting.

18. The non-transitory computer-readable device of claim 11, further comprising:
when the x-ray tube is aligned with the x-ray sensor head, capturing a first x-ray image by an x-ray capturing element affixed to the x-ray sensor head.

19. The non-transitory computer-readable device of claim 11, wherein the pending position of the x-ray sensor head is determined to occur when the x-ray sensor head is in a constant position for a period of time larger than a threshold.

20. A dental system, comprising:
an x-ray sensor head, comprising:
a first image sensor configured to capture a first image, the first image captured at an origin position;
a memory, and
one or more processors coupled to the memory, wherein the one or more processors are configured to:
determine a pending position of the x-ray sensor head with reference to the origin position;
determine whether the first image shares a common axis of reference with an x-ray tube based on a correlation between the first image and a second image captured by a second image sensor affixed to the x-ray tube;
calculate a position of the x-ray tube with reference to the pending position of the x-ray sensor head and the common axis of reference; and
based on the position of the x-ray tube, determine whether the x-ray tube is perpendicularly aligned towards the x-ray sensor head when at the pending position.
